(19) United States
(12) Patent Application Publication
(10) Pub. No.: US 2022/0160044 A1
(43) Pub. Date: May 26, 2022

(54) Smart Electronic Mask, Headset and Inhaler

(71) Applicant: Cedarz Group, LLC, Sachse, TX (US)

(72) Inventor: Roland J. Moubarak, Sachse, TX (US)

(73) Assignee: Cedarz Group, LLC, Sachse, TX (US)

(21) Appl. No.: 17/529,415

(22) Filed: Nov. 18, 2021

Related U.S. Application Data
(63) Continuation-in-part of application No. 17/489,860, filed on Sep. 30, 2021.
(60) Provisional application No. 63/198,973, filed on Nov. 26, 2020.

(51) Int. Cl. A24F 40/53; A24F 40/10; A24F 40/65; A24F 40/95; A24F 40/51; A24F 40/46; A61M 11/04; A24F 40/60; A62B 18/02

(52) U.S. Cl. CPC A24F 40/53; A24F 40/10; A24F 40/65; A24F 40/95; A24F 40/51; A61M 2205/8206; A61M 11/042; A24F 40/60; A62B 18/025; A61M 2205/3592; A24F 40/46

(57) ABSTRACT

Provided is a smart inhaler, headset and wearable smart mask configured to operate as an air ionizer and a controlled multi-liquid atomizer. The smart mask, headset and smart inhaler use a mature technology to deliver various types of liquid solutions to different applications from health care, drug delivery, immunization to recreational and gaming uses. In addition, the smart mask and headset contain multiple actuators to provide haptic feedback to the areas around the mask and headset. The headset, smart mask and inhaler have a direct communication path to a smart device or it can be a smart device by itself; its various environmental and gas sensors act as a feedback mechanism. The detection level of the organic and non-organic VOC gas emitted when exhaling can be communicated and stored for analysis purposes. The detected gas can be regenerated on the same smart mask or a different remote smart mask.

### CROSS-REFERENCES TO RELATED APPLICATIONS

The present application claims the benefit of the filing date of an application entitled “Smart Electronic Mask and Inhaler,” application Ser. No. 17/489,860, filed Sep. 30, 2021, assigned to the assignee of the present application, and herein incorporated by reference; which claims the benefit of the filing date of a provisional application entitled:

“Smart Electronic Mask and Smart Electronic Inhaler” Ser. No. 63/198,973, filed Nov. 26, 2020, assigned to die assignee of the present application, and herein incorporated by reference.

### FIELD OF THE DISCLOSURE

The claimed subject matter relates generally to wearable smart mask and smart inhaler, more specifically, to a smart inhaler and wearable smart mask that may be utilized for gaming or as a smart inhaler.

### BACKGROUND

Current technology in the area of wearable face masks and headsets does not provide for air ionization and solution aerosolization to stimulate smell and taste sensations and to judder areas around the head, including the mouth and ears, using haptic actuators. Current masks and headsets cannot control the delivery of a liquid substance to the mouth and nostril areas nor act as smart inhaler in the medical and immunization market. In addition, current technology cannot act as a smart controlled vaping replacement for the recreational market, controlling an amount of substance delivery like nicotine and CBD to prevent overdosing.

### SUMMARY

The claimed subject matter relates to air ionization and solution aerosolization to stimulate smell and taste sensations and to judder the area around the mouth by using haptic actuators. One goal is to introduce a new gaming device to stimulate the smell, taste and skin sensory around the mouth and create a new market that can be considered the 4^{th} or 5^{th} dimension for the video gaming market. This device can play a major role in multiple other markets as well. Since the mask can control the delivery of a liquid substance to the mouth and nostril areas, it can act as smart mask or smart inhaler in the medical and immunization market. In addition, it can act as a smart controlled vaping replacement for the recreational market, where it can control the amount of substance delivery like nicotine and CBD to prevent overdosing. Another market that this device can reach is the adult sensual market.

Provided is an apparatus for a wearable mask, or respirator, that can be configured to operate as an air ionizer and a controlled multi-liquid atomizer. In short, the claimed subject matter introduces a new concept to the market: new types of masks and inhalers that use a mature technology to deliver various types of liquid solutions to different applications from health care, drug delivery, immunization to recreational and gaming uses.

In addition, the smart mask contains multiple actuators to provide haptic feedback to the areas around the mask. The mask has a direct communication path to a smart device or it can be a smart device by itself; its various environmental and gas sensors act as a feedback mechanism. The detection level of the organic and non-organic VOC gas emitted when exhaling can be communicated and stored for analysis purposes. The detected gas can be regenerated on the same mask or a different remote smart mask.

This system is envisioned to work with other smart vital monitoring devices like a smart watch to achieve a closed loops configuration for the delivery of the necessary atomized solution to the user.

In the disclosed technology illustrated in FIGS. 1-17 the drawings are not up to scale nor do they represent the exact shape and function location of each device or interface.

The invention can be divided into four separate sub-systems:
1—A controller that communicates to a smart device and controls the functions below.
2—An atomizer that delivers one or more liquid solutions into the breathing chamber.
3—An ionizer that charges the inhaled air to create a fresh, smog sense and purify the air.
4—Haptic actuator(s) that can deliver multi-dimensional tactical sensations.

The Electronic smart breathing mask and headset system, described herein, is configured to hold a removable container of liquid solution to be used for medicinal or recreational purposes. The mask and headset are smart devices with sensors and actuators that can control and activate the delivery of the solution held in the container when needed or as needed. In addition to stimulating the smell sensation via air ionization and liquid atomization, the mask includes a haptic element(s) to stimulate the lower facial region.

In addition, the smart mask and headset can be configured to connect to a smart device via a wireless interface. Thus, the mask and headset can act as a smart peripheral to a smart phone, gaming console or to an external network; the mask and headset can also include optional sensors and peripherals to connect to the cloud as an Internet of Things device. This device is envisioned to be an apparatus to the mouth; it provides a controlled delivery system, as needed or by a protocol, for prescription or non-prescription drugs, recreational substances. It can also provide a new type of peripheral far the video gaming industry. The smart electronic inhaler is a sub category of the smart mask; it can play a big role in the medical and recreational markets.

This summary is not intended as a comprehensive description of the claimed subject matter but, rather, is intended to provide a brief overview of some of the functionality associated therewith. Other systems, methods, functionality, features and advantages of the claimed subject matter will be or will become apparent to one with skill in the art upon examination of the following figures and detailed description.

### BRIEF DESCRIPTION OF THE DRAWINGS

In the following brief description, reference is made to accompanying drawings, and specific embodiments in which the invention may be used are shown by way of illustration. It is to be understood, however, that other embodiments may be utilized and that various changes may be made without departing from the spirit and scope of the present invention. The following description is, therefore, not to be taken in a limiting sense.

FIG. 1 is an illustration of a front view of a wearable electronic mask in accordance with the claimed subject matter.

FIG. 2 is an illustration of a side view of the wearable electronic mask of FIG. 1.

FIG. 3 is an illustration ofacontroller module of the wearable electronic mask of FIGS. 1 and 2 in greater detail.

FIG. 4 illustrates an interchangeable cartridge, first introduced in FIG. 2 in greater detail.

FIG. 5 is an illustration of an interchangeable cartridge being inserted into an embodiment of the claimed subject matter.

FIG. 6 is an illustration of the front of the controller module.

FIG. 7 is an illustration of a side view of wearable mask of FIGS. 1 and 2.

FIG. 8 is an illustration of an ionization chamber, first introduce in FIG. 2 is greater detail.

FIG. 9 is an illustration of the ionization chamber, first introduce in FIG. 2 and shown in FIG. 8, is greater detail.

FIG. 10 is an illustration of a haptic actuator, first introduced in FIG. 2, is greater detail.

FIG. 11 is an illustration of the wearable mask of FIGS. 1 and 2, showing some internal components.

FIG. 12 is an illustration of the wearable mask of FIGS. 1 and 2 as part of a headphone set.

FIG. 13 illustrates the mask of FIG. 12 as worn by a person.

FIG. 14 is an illustration of a side view of a smart inhaler.

FIG. 15 illustrates a smart inhaler subsystem of the smart inhaler of FIG. 14.

FIG. 16 illustrates a cartridge cover, first introduced in FIG. 7, for the mask and inhaler of FIGS. 14 and 15.

FIG. 17 illustrates one embodiment of the controller, introduced with FIGS. 1-3 and for the mask and inhaler of FIGS. 14 and 15.

FIG. 18 illustrates one embodiment of a wearable headset that incorporates a nostril atomizer.

FIG. 18 illustrates the nostril atomizer and supporting hardware of FIG. 18 in greater detail.

### DETAILED DESCRIPTION OF THE FIGURES

Although described with particular reference to a wearable electronic mask, the claimed subject matter can be implemented in any application in which controlled delivery of a liquid substance, air (gas) ionization and/or haptic feedback is desirable. Those with skill in the relevant arts will recognize that the disclosed embodiments have relevance to a wide variety of environments in addition to those described below. In addition, the methods of the disclosed technology can be implemented a combination of software and hardware. The hardware portion can be implemented using specialized logic; the software portion can be stored in a memory and executed by a suitable instruction execution system such as, but not limited to, a microprocessor, personal computer (PC), smartphone or cloud computing.

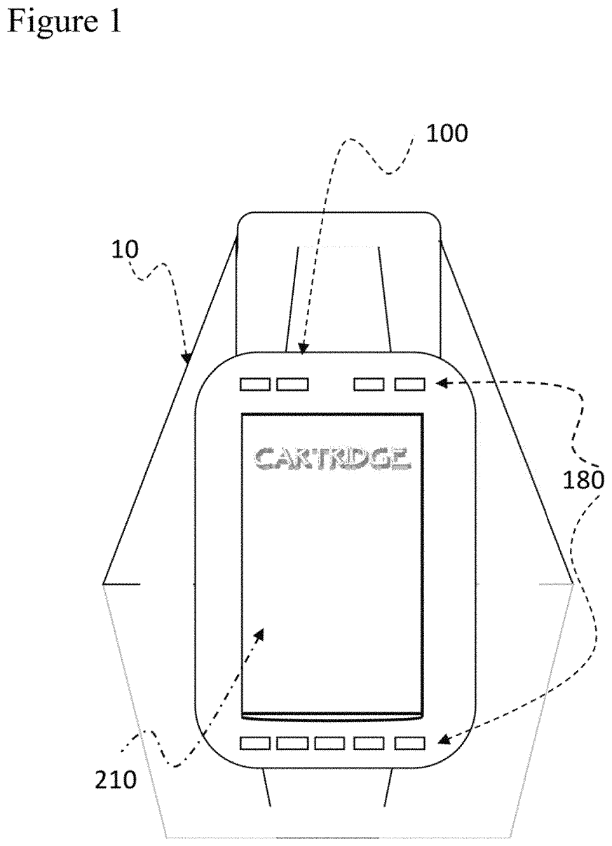

FIG. 1 is an illustration of a front view of a wearable electronic mask 10 in accordance with the claimed subject matter. It should be understood that mask 10 is not limited by the shape as illustrated in FIG. 1. Mask 10 includes a controller module, or simply “controller,” 100. Controller 100 is powered by a chargeable battery (not shown) that may be charged via a USB port (not shown). Controller 100 also includes a processor (not shown) that interfaces with a wireless interface module (not shown) for connection to another device such as, but not limited to, a network, smart device or gaming console, and a voltage booster (not shown). Controller 100 includes multiple light emitting diodes (LEDs) 180 that indicate various functions. Attached to the front of mask 10 is an interchangeable cartridge 210, which stores a liquid solution.

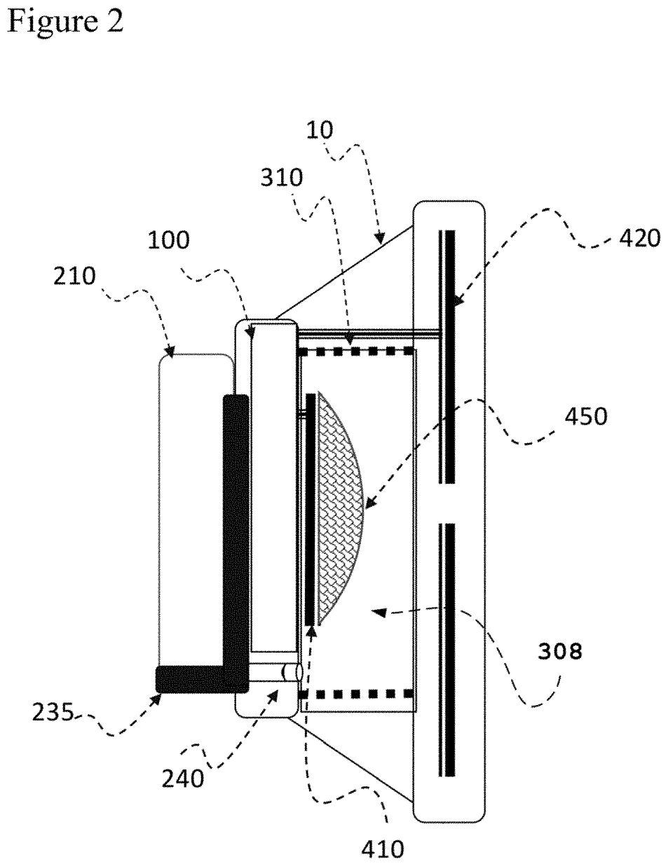

FIG. 2 is an illustration of a side view of the wearable electronic mask 10 of FIG. 1 showing additional detail. FIG. 2 includes mask 10, controller 100 and interchangeable cartridge 210, all introduced above in conjunction with FIG. 1. Interchangeable cartridge 210 is connected to controller 100 via a cartridge holder 235 and is attached to a liquid mixer 240 where an actuator, such as, but not limited to, an Inkjet type Piezoelectric transducer or heating element, controls the flow of the liquid solution in interchangeable cartridge 210. An internal ionization chamber 308 isolates breathed air from outside air. Internal ionization chamber 308 is the area within an ionization membrane 310 and contains a haptic actuator 410. The haptic set of actuators, which include haptic actuator 410 and a haptic actuator 420 may judder the mouth, lips and tongue areas to simulate such simulation as wind, shock and grabbing sensations. Actuator 410 is covered by a custom silicon shape 450 related to the game played. The array of actuators 410 and 420 can deliver a vibrational sensation on the face and mouth area. This feature can be used in the gaming and adult toy industries.

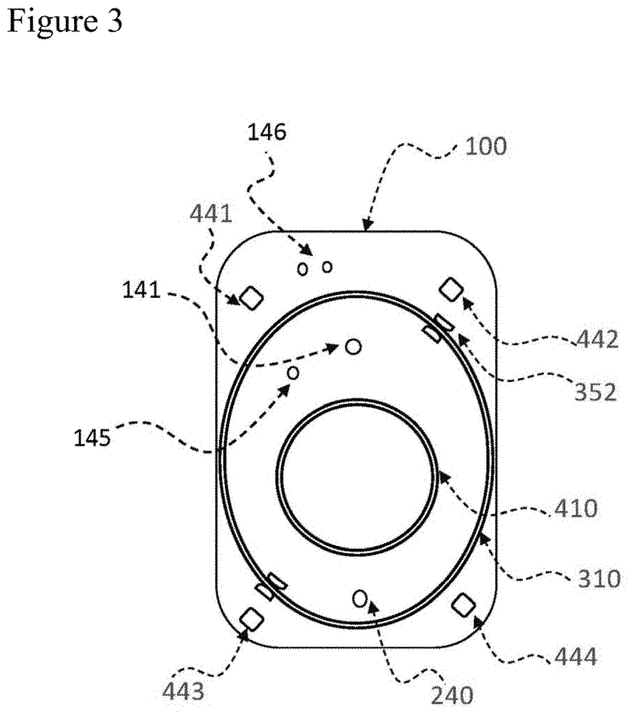

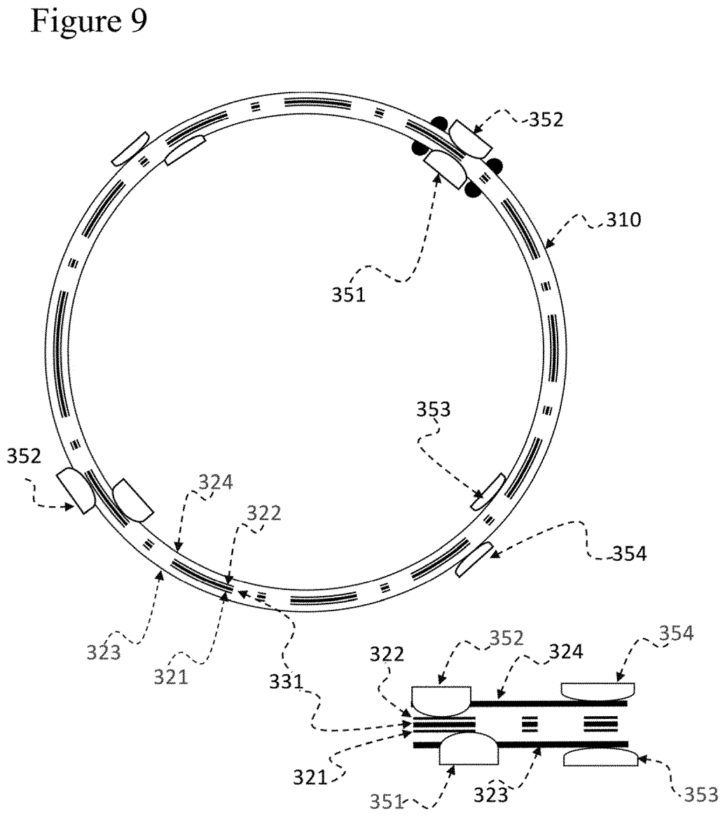

FIG. 3 is an illustration of one side of controller 100 of wearable electronic mask 10 of FIGS. 1 and 2 in greater detail. Controller 100 illustrated is connected via a wireless interface (not shown) to a network (not shown), a smart device (not shown) or a gaming console (not shown). In the illustrated embodiment, controller 100 is powered by a chargeable battery (not shown) that is charged via a USB port (not shown). Controller 100 consists of a processor (not shown) that interfaces to the wireless interface, a voltage booster (not shown) to drive multiple piezo elements (not shown) via connectors 441, 442, 443, 444 and actuator 410 (FIG. 2). In addition, high voltage that is generated drives an ionization element (not shown) on board via 351, 352, 353 and 354 connections (see FIG. 9). The system is a simplified small inkjet printer controller that is geared toward delivering a controlled amount of a liquid residing in interchangeable cartridge 210 and connected to controller 100 via cartridge holder 235 (FIGS. 2 and 3).

FIG. 3 also shows environmental, or breath, sensor 145, liquid mixer 240 (FIG. 2), internal ionization chamber 310 (FIG. 2) and a micro electronic mechanical system (MEMS) microphone 141.

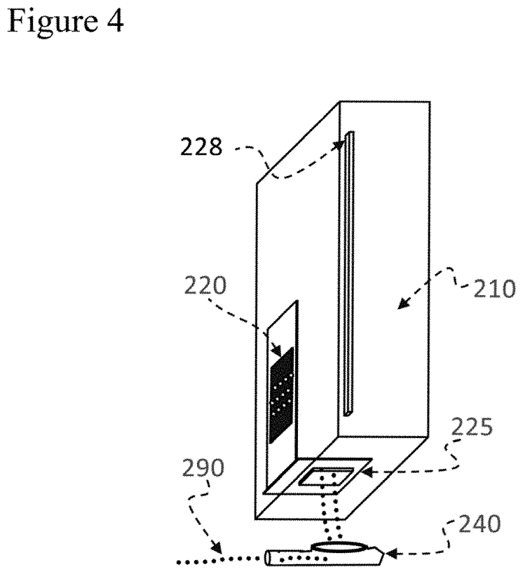

FIG. 4 is an illustration of interchangeable cartridge 210, first introduced in FIG. 2 in greater detail. FIG. 4 includes liquid mixer 240, introduced above in conjunction with FIGS. 2 and 3. An atomizer function may be derived from a well mature Inkjet market that delivers a small controlled quantity or a concentrated liquid that can be used in different markets. Using this technology gives us access to the Inkjet cartridge market where the liquid content could be one or more types of independent solutions.

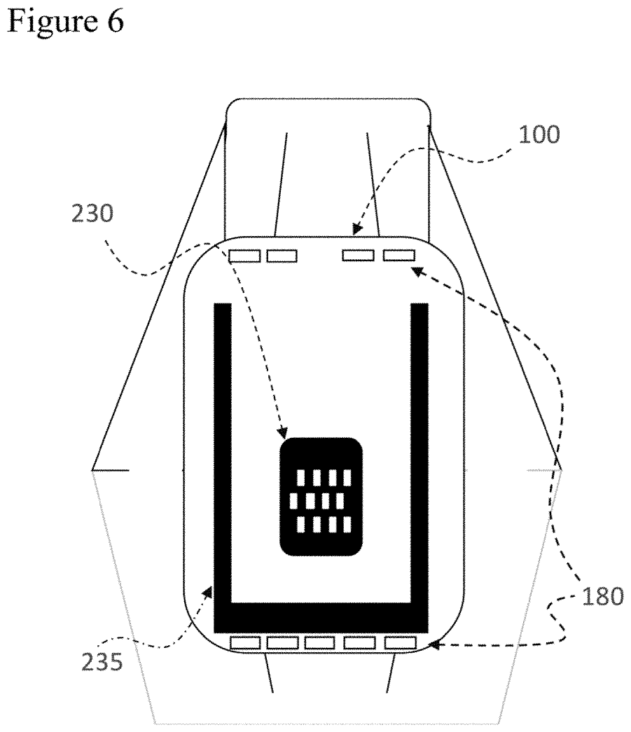

This mature technology will allow the usage of a cartridge connector interface 220 and the piezo electric transducer 225 for precise solution delivery. In addition, an identity memory (not shown) is built into the cartridge protocol. This complex liquid delivery system, or piezo electric transducer or heating element 225, which feeds into mixer 240, is made accessible by the sheer volume of inkjet market. The liquid mixing of the various solutions in mixer 240 is delivered to an inhalation chamber 290. Solution Cartridge 210 is affixed to controller 100 by a mechanism where a rail 228 slides into groves in cartridge holder 235 (FIGS. 2 and 3), allowing connector 220 to make contact with a socket 230 (see FIG. 6).

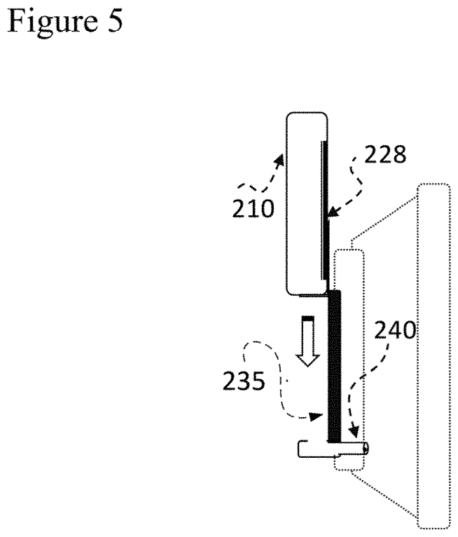

FIG. 5 is an illustration of interchangeable cartridge 210 (FIGS. 1 and 2) being inserted into cartridge holder 235 (FIG. 2) of the claimed subject matter. A mechanism 228 on cartridge 210 slides into groves (not shown) in cartridge holder 235. Also shown is liquid mixer 240 (FIGS. 2-4).

FIG. 6 is an illustration of the front of controller module 100 (FIGS. 1-3), including LEDs 180 (FIG. 1) and cartridge holder 235 (FIGS. 2, 5 and 6). Also shown in FIG. 6 is a socket 230 that connects to connector 220 (FIG. 3) when interchangable cartridge 210 (FIGS. 1 and 2) is inserted into cartridge holder 235.

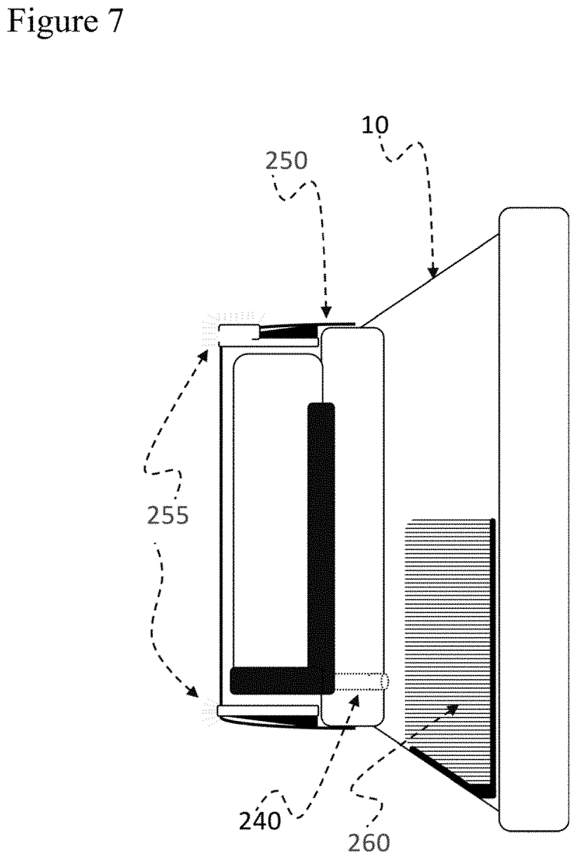

FIG. 7 is an illustration of a side view of wearable mask 10 of FIGS. 1 and 2. FIG. 7 includes liquid mixer 240 (FIGS. 2-4). A cartridge cover 250 is illustrated but not limited to this type of cover. Light tubes 255 enable LEDs 180 (FIGS. 1 and 7) to be seen when cover 250 is installed. Also shown is a vent opening 260 that points downward.

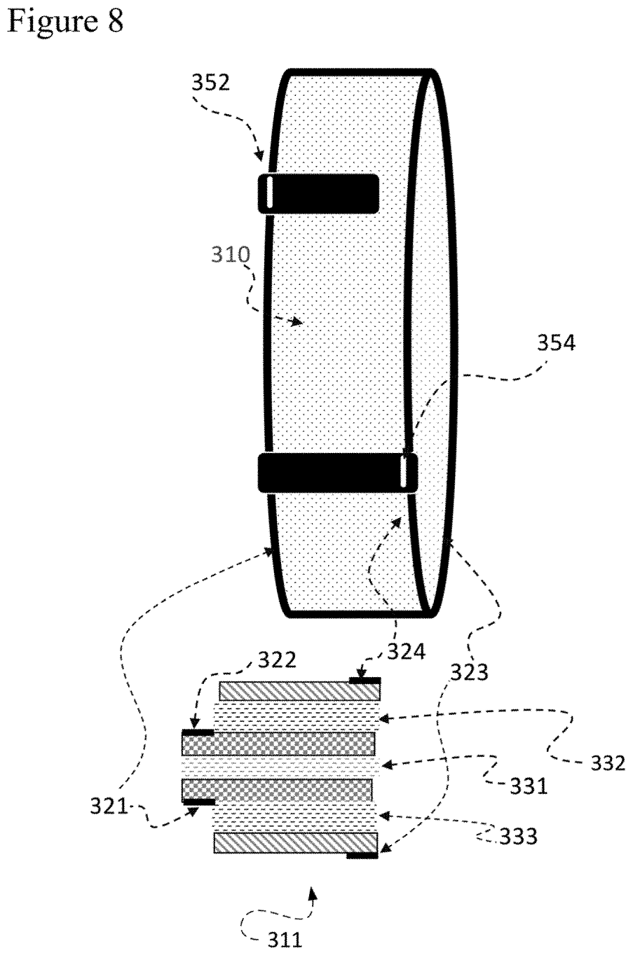

FIG. 8 is an illustration of a ionization chamber 310, first introduced in FIG. 2 is greater detail and layers of material in a filter type ionizing element 311 that comprise one embodiment of ionization chamber 310. Also shown are connections 352 and 354 (FIG. 3). Filter type ionizing element 311 consists of multiple conductive layers 2 more layers made from, but not limited to, conductive material like silver fabric, silver plating, multilayer flexible PCB or similar material separated by an isolated layer 331 where an electric field can be generated to ionize the air between layers 321 and 322. Exterior layers 323, 324 are driven at a lower voltage with respect to the skin of the user for safety reasons. Interior layers 331-333 are also shown. Breathableionization creates a fresh smell and can disinfect various types of microorganism that are airborne.

FIG. 9 is an illustration of a top and side view of ionization chamber 310, shown in FIGS. 2 and 8, is greater detail. As shown in FIG. 8, ionization chamber 310 includes layers 321 and 322 (FIG. 8), exterior layers 323 and 324 (FIG. 8), isolation layer 331 (FIG. 8), connections 352 and 354 (FIGS. 4 and 8) and additional connectors 351 and 353.

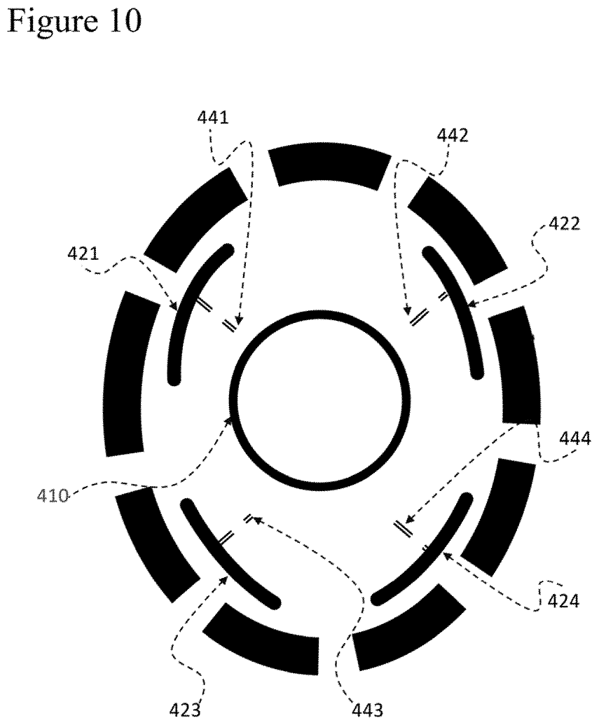

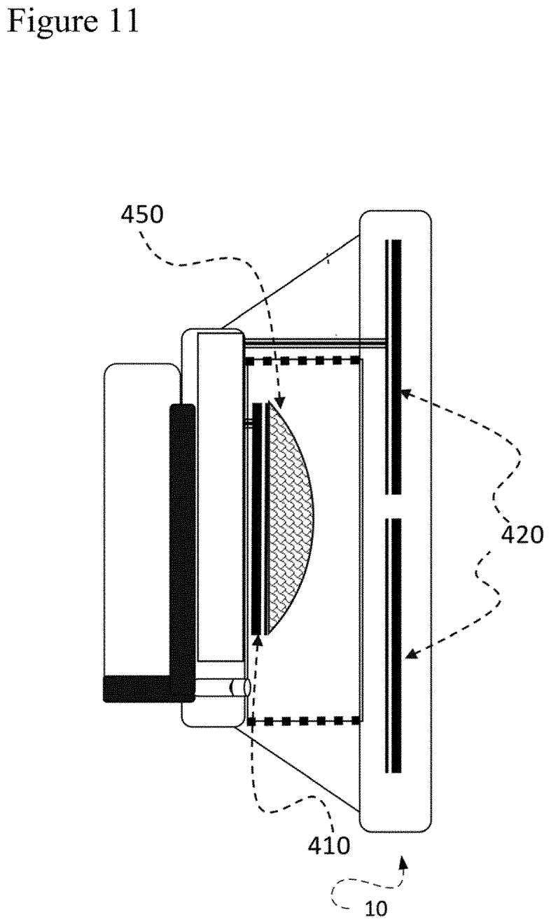

FIG. 10 is an illustration of haptic actuator 410, first introduced in FIG. 2, is greater detail. Also included are peripheral actuators 421-424. Haptic Actuator 410 can deliver tactile sensations in the center of the mask. FIG. 10 also shows connectors 441-444 (FIG. 3). Actuator 410 is covered by custom silicon shape 450 (FIG. 2, see FIG. 11), related to the game played. The array of actuators can deliver a vibrational sensation on the face and mouth area. This feature can be used in the gaming and adult toy industries.

FIG. 11 is an illustration of wearable mask 10 of FIGS. 1 and 2, showing some internal components. Included in FIG. 11 are haptic actuator 410 (FIG. 10), haptic actuators 420 (FIG. 2) and custom silicon shape 450 (FIGS. 2 and 10).

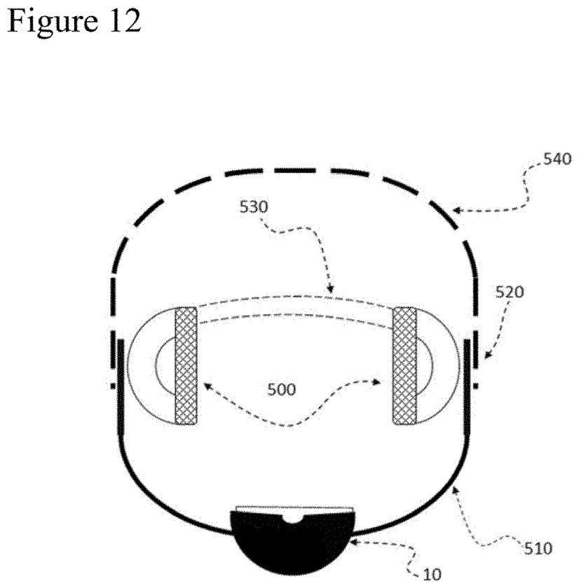

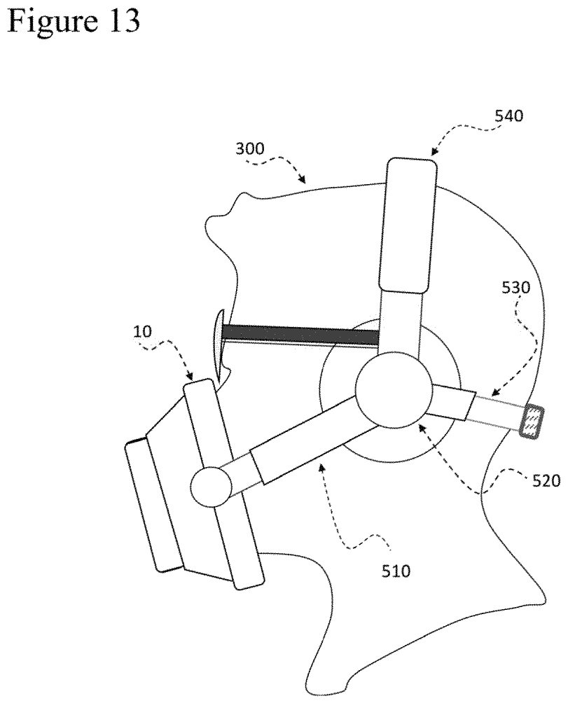

FIG. 12 is an illustration of the wearable mask 10 of FIGS. 1 and 2 as part of a headphone set (see FIG. 13). Mask 10 connects to a classic ear-muffed headphone over ears 500 that has an over the head strap 540. An additional behind the head strap 530 may be used to keep headphone in place and support an adjustable mask connection 510 to ensure that mask 10 stays in position. The three connections 510, 530 and 540 have a hinge 520 across the ear piece(s) so that it can be folded and stowed.

FIG. 13 illustrates mask 10 of FIG. 12 as worn on a head 300 of a person. Shown are straps, or connections 510, 530 and 540, and hinge 520, all introduce above in conjunction with FIG. 13. Gaming mask cartridge 210 (FIGS. 2-5) may hold one or multiple primary odors/tastes to generate different smells during a game. For example, when a player is swimming in a video game, a salty solution can be injected to make them smell and taste the ocean. If a player is shooting, we can deliver a gun powder smell and so on. In addition, the haptic function and ionization augments the sensational experience. Thus, this disruptive device to the gaming market includes mask-to-mask communications gaming using latest technologies like 5G technology, blue tooth mesh, Zigbee NFC technologies which allow a gamer to communicate and interface directly to other gamer using voice commands or other control platforms. As such, it will introduce a new dimension to the gaming industry.

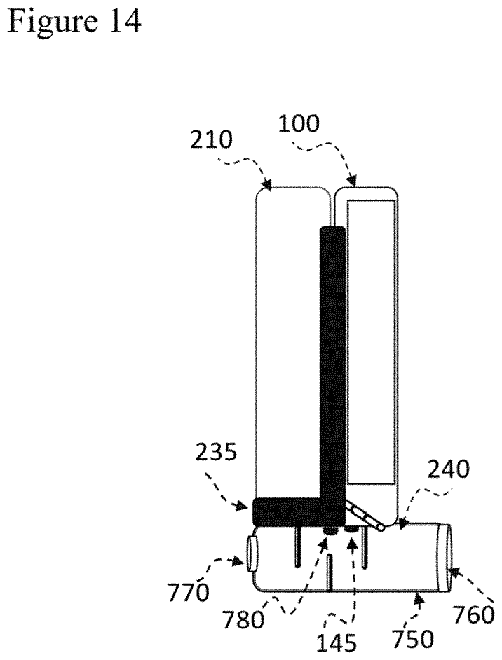

FIG. 14 is an illustration of a side view of a smart inhaler. FIG. 14 includes controller 100, interchangeable cartridge 210, cartridge holder 235 and liquid mixer 240, all illustrated above in conjunction with FIG. 2.

Also includes are an environmental, or breath, sensor 145 that functions as a feedback mechanism in addition to the output of liquid mixer 240 where an actuator controls the flow of the liquid solution stored in interchangeable cartridge 210.

The Smart Electronic Inhaler is envisioned to be a sub-system of mask 10 or stand-alone inhaler where the core parts are similar except that liquid solution delivery 240 is fed to an inhaling channel 750. Inhaling channel 750 has a mouthpiece opening 760 and an air inlet opening 770 in addition to a Breath sensor 145 to detect volatile organic compounds (VOC) and an ultraviolet (UV) LED 780 to insure sterilization of the channel.

A list of various VOCs will be measured depending on the availability of the sensors in the market. The List of VOCs includes but is not limited to the following: Acrylonitrile, Propanal, Tetradecane, Octanal, Dipropoxypropane, Methyl Methacrylate, Pentane, Xylene, Hexanal, Nonanal, Nitric Oxide, Trimethylamine (TMA), Pentene, Nitrogen Dioxide, Isoprene, Isopropanol, 2-Propanol, Acetaldehyde, Benzene, Methane, Ethane, Ethanol, Hydrogen Sulfide and Acetone.

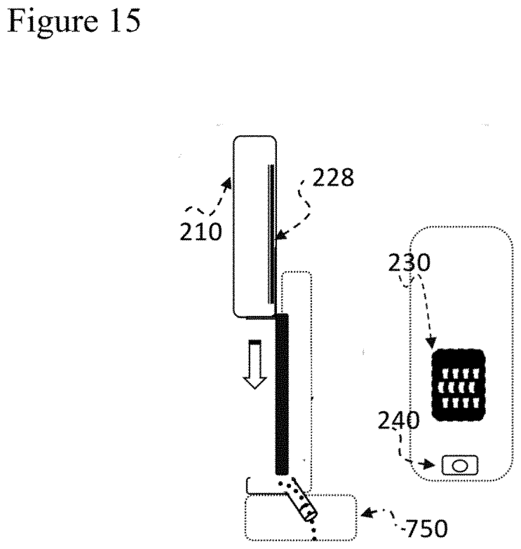

FIG. 15 illustrates a side and front view of a smart inhaler subsystem of wearable mask 10 of FIG. 14 or a stand-alone inhaler. FIG. 15 includes cartridge 210 (FIG. 14), rail 228 (FIGS. 4 and 5), socket 230 (FIG. 6) and liquid mixer 240 (FIG. 14). The Smart Liquid solution delivery mixer 240 is fed to an inhaling channel 750.

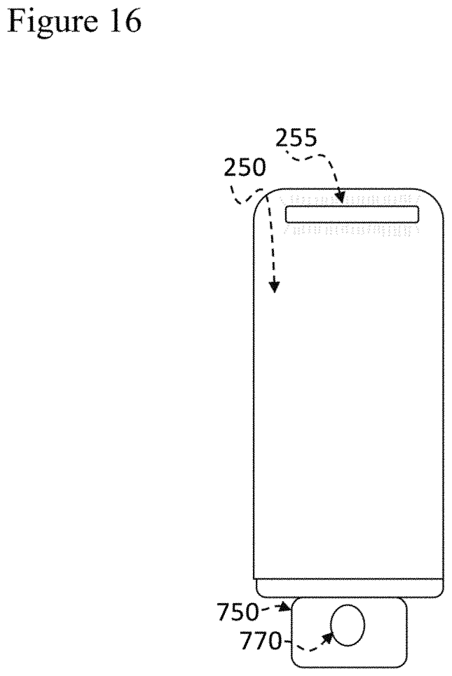

FIG. 16 illustrates cartridge cover 250 with light tubes 255, both introduced above in conjunction with FIG. 7. Also shown are inhaling channel 750 (FIG. 15). Inhaling channel 750 includes an air inlet opening 770.

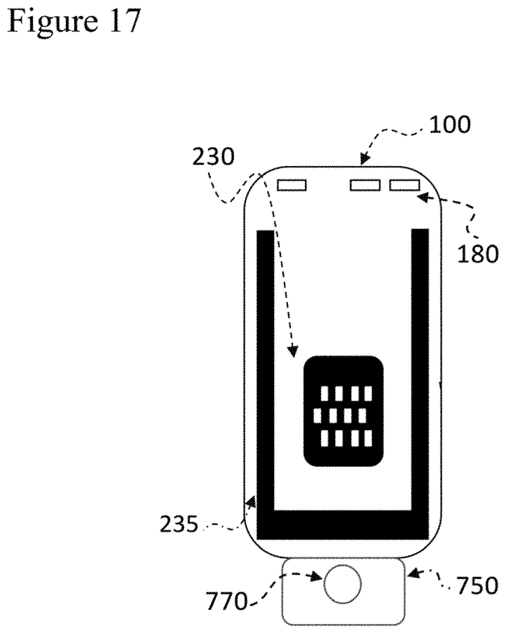

FIG. 17 illustrates one embodiment of controller 100, introduced in conjunction with FIGS. 1-3, 6 and 14. Also shown are socket 230 (FIGS. 6 and 15), LEDs 180 (FIGS. 1 and 6), cartridge holder 235 (FIGS. 2, 6, 7 and 14), inhaling channel 750 (FIGS. 15 and 16) and inlet opening 770 (FIG. 16).

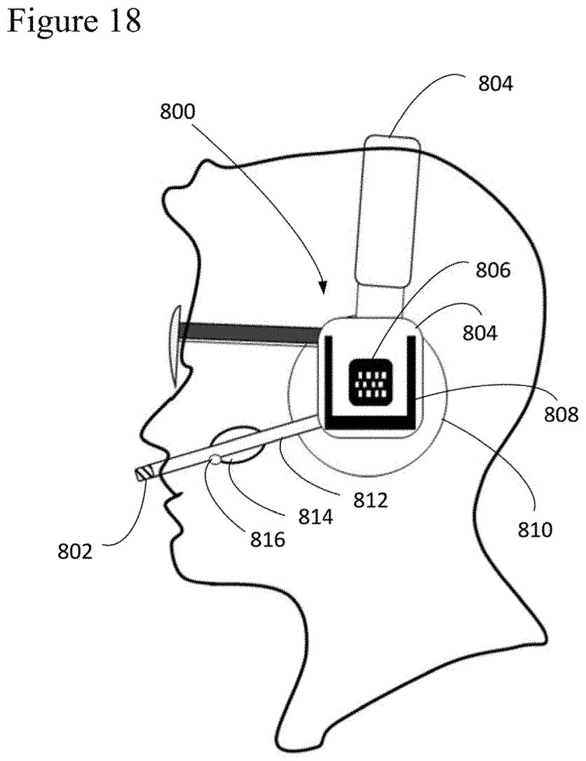

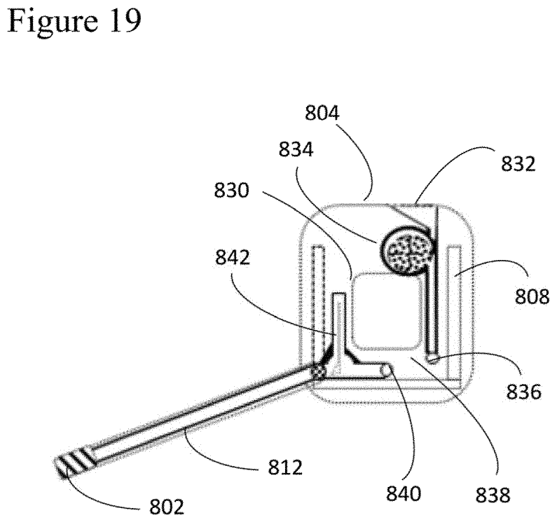

FIG. 18 illustrates one embodiment of a wearable headset 800 that incorporates a nostril atomizer 802. Headset 800 includes a head strap 804, similar to head strap 540 shown in FIGS. 12 and 13, a controller 804, a socket 806, similar to socket 230 of FIGS. 6, 15 and 16, a cartridge rail 808, similar to cartridge rail 235 of FIGS. 2, 5, 6, 14 and 17, and an ear piece 810. Ear piece may include haptic actuators (not shown) like 420 and 430, described above in conjunction with FIGS. 2 and 11 to judder a user's ear. Controller 804 is coupled to an arm 812. Arm 812 includes a piezo electric actuator 814, a microphone 816 and nostril atomizer 802. The function of controller 804, arm 812 and nostril atomizer 802 are described below in conjunction with FIG. 19.

FIG. 19 illustrates nostril atomizer 802, arm 812 and internal components of controller 804 of FIG. 18 in greater detail. Controller 804 includes cartridge rail 808, an interchangeable cartridge 830, similar to interchangeable cartridge 210 of FIGS. 1, 2, 4-6 and 13-15, an outside air intake 832, an air pump 834 and an air outlet 836.

Like with interchangeable cartridge 210, an atomizer function may be derived from a well mature Inkjet market that delivers a small controlled quantity or a concentrated liquid that can be used in different markets. Using this technology gives us access to the Inkjet cartridge market where the liquid content could be one or more types of independent solutions. This mature technology will allow the usage of a cartridge connector interface, like interface 220 (FIG. 4) and a piezo electric transducer, like transducer 225 (FIG. 4), for precise solution delivery. In addition, an identity memory (not shown) may be built into the cartridge protocol. This complex liquid delivery system, or piezo electric transducer or heating element, which feeds into a mixing chamber 838, is made accessible by the sheer volume of inkjet market. The liquid mixing of the various solutions in mixing chamber 838 is delivered to air outlet 840 and to nostril atomizer 802. Interchangeable Cartridge 830 is affixed to controller 804 by a mechanism where a rail (not shown) slides into cartridge rail 808, allowing a connector to make contact with socket 806 (FIG. 18).

Outside air is taken in via outside air intake 832 and forced through air outlet 836 into a mixing chamber 838 by air pump 834. In mixing chamber 838, the air is mixed with a solution, such as a fragrance or perfume, stored in an interchangeable cartridge 830. The air with an added fragrance is then forced through an air outlet 840 in arm 812. An ionizer 842 coupled to arm 812 charges the air to create a fresh, smog sense and purify the air. The air, including fragrance, is then forced out through nostril nozzle 802 where it is inhaled by a user. It should be understood that headset 800 may be combined with the technology of mask 10 (FIGS. 1, 2 and 7) to produce a gaming system that stimulates multiple senses, thereby creating a more realistic experience.

The recreational substance market can benefit greatly from a controlled cartridge substance delivery method in accordance with the disclosed technology. In one delivery method, an intelligent device that can be part of an Internet of Things family connected to the cloud or a smart device (not shown) can control the delivery of the recreational substance. This delivery method can ensure that the solution used is certified and trackable via a sealed cartridge; also, it can prevent overdosing. The system may include multiple sensors to measure the exhaled air to prevent overdosing and to help in quitting a substance addiction over a profile that can be monitored and controlled remotely. In addition, this device can interface to wearable devices to measure vitals, etc. This can play a disruptive role in the delivery of nicotine based or cannabis-based markets.

The recreational market can benefit from the smart electronic mask or from the smart inhaler to deliver controlled substance in a controlled platform like Smart Fume.

The medical drug delivery market may also benefit greatly from the delivery methods of the disclosed technology. An intelligent device that can be part of an Internet of Things family that is connected to the cloud or a smart device can control the delivery of one or multiple drugs. A prescribed “Drug Cartridge” may be traceable by a unique ID and can be administered by a physician remotely via the cloud. In addition, the system may have multiple sensors to measure different types of inhaled and exhaled air as well as temperature; moreover, it can interface to wearable devices to measure vitals, etc. We envision this feature to be very useful with young, elderly, disabled and high-risk patients.

The Medical smart mask is envisioned to have multiples VOC, Carbon Dioxide and temperature sensors which serve as a feedback mechanism for the delivery of the medication needed. The Multi VOC sensors with other control mechanism, that could be local to the mask or external, can serve as a trigger to an event like the delivery of necessary medication. The Medical smart inhaler will have a subset of the features of the smart medical mask. For example, if the user is using a Medical smart mask for Asthma condition, the mask will rely on the measurement of the Nitric Oxide Organic Volatile compound that is directly associated with the airway inflammation. The algorithm can notify the person, a guardian or a monitoring system and can release an asthma medication to reduce the symptoms. The smart inhaler can be used also similar to the smart mask.

The family of intelligent devices can be used as a vaccination delivery method which introduces a controlled amount of inactivated pathogens load, such as virus, bacteria, renominate protein(s) and fungi, over a period of time while monitoring vital feedback from the on board and off board sensors. In addition, this device can be used in allergy treatment where a small portion of allergens can be used and the profile can be monitored by a protocol and adjusted remotely by the physician.

This system will revolutionize the pharmaceutical industry through monitoring, securing, and controlling prescribed drugs. In high risk patients, this system might become a necessity and will prevent overdose or under dose scenarios. The customized or controlled substance can be loaded into a medical cartridge where it can contain more than one prescription. So, the new pharmacy model will replace your prescription cartridge so you can use it with your electronic mask or electronic inhaler for precise timing and dose delivery.

While the claimed subject matter has been shown and described with reference to particular embodiments thereof, it will be understood by those skilled in the art that the foregoing and other changes in form and detail may be made therein without departing from the spirit and scope of the claimed subject matter, including but not limited to additional, less or modified elements.

We claim:

1. A smart electronic head set, comprising:
a head set;
a cartridge holder coupled to head set;
a cartridge adapted to fit into the cartridge holder;
an outside air intake;
a mixing chamber adapted to mix air from the outside air intake with a liquid from the cartridge; and
a handset arm, the handset arm comprising a nostril nozzle;
wherein the air mixed with the liquid is delivered to a user via the nostril nozzle.

2. The smart electronic head set of claim 1, further comprising a plurality of haptic actuators coupled to the head set.

3. The smart electronic head set of claim 1, wherein the atomizer is based upon an inkjet cartridge technology.

4. The smart electronic head set of claim 1, wherein the atomizer is a piezo electric transducer.

5. The smart electronic head set of claim 1, further comprising an ionizer in the handset arm configured to insure sterilization of the air mixed with the liquid.

6. The smart electronic head set of claim 1, wherein the liquid is a fragrance.

7. The smart electronic face mask of claim 1, wherein the liquid is a medicine.

8. A smart electronic gaming device, comprising:
a head set;
a cartridge holder coupled to head set;
a cartridge adapted to fit into the cartridge holder;
an outside air intake;
a mixing chamber adapted to mix air from the outside air intake with a liquid from the cartridge;
a handset arm, the handset arm comprising a nostril nozzle;
wherein the air mixed with the liquid is delivered to a user via the nostril nozzle;
a face mask;
an inhalation chamber coupled to the face mask;
a controller module coupled to the inhalation chamber;
a cartridge holder;
an interchangeable cartridge configured to be inserted into the cartridge holder; and
an atomizer configured to deliver contents of the interchangeable cartridge into the inhalation chamber;
wherein the controller module controls delivery of the contents of the interchangeable cartridge into the inhalation chamber.

9. The smart electronic gaming device of claim 8, further comprising a plurality of haptic actuators coupled to the head set.

10. The smart electronic gaming device of claim 8, wherein the atomizer is based upon an inkjet cartridge technology.

11. The smart electronic gaming device of claim 8, wherein the atomizer is a piezo electric transducer.

12. The smart electronic gaming device of claim 8, further comprising an ionizer in the handset arm configured to insure sterilization of the air mixed with the liquid.

13. The smart electronic gaming device of claim 8, wherein the liquid is a fragrance.

14. The smart electronic gaming device of claim 8, wherein the liquid is a medicine.
